(19) United States
(12) Patent Application Publication
(10) Pub. No.: US 2017/0087106 A1
(43) Pub. Date: Mar. 30, 2017

(54) Method for supporting gut health

(71) Applicant: Ivo Pischel, Rossbach (DE)

(72) Inventor: Ivo Pischel, Rossbach (DE)

(21) Appl. No.: 14/868,447

(22) Filed: Sep. 29, 2015

(51) Int. Cl. A61K 31/19; A61K 35/741; A61K 31/70

(52) U.S. Cl. CPC A61K 31/19; A61K 31/70; A61K 35/741

(57) ABSTRACT

Calcium pyruvate and derivatives or formulations thereof can be employed for supporting gut health and lessen inflammations, for example in chronic inflammatory diseases of the digestive tract, and irritable bowel syndrome. Therefore, the invention relates to the use of Calcium pyruvate preparations for treating inflammatory diseases, especially of the digestive tract of humans and mammalian animals, pets and livestock. The digestive tract includes the oral cavity, the pharynx, the esophagus, and the gastrointestinal tract. In one embodiment, said inflammatory disease is an irritable bowel syndrome (IBS) or an inflammatory bowel disease (IBD). In another embodiment, the inflammatory diseases are selected from the group of gingivitis, parodontitis, pharyngitis, esophagitis, gastritis, lymphocytic colitis, ulcerative colitis, diverticulitis, duodenitis, and Crohn's disease.

### TECHNICAL FIELD OF THE INVENTION

The invention relates to a method of applying a food, a dietary supplement or a drug composition comprising an active ingredient, preferably calcium pyruvates and more preferably calcium pyruvate monohydrate (CPM) or functionally active derivative and combinations or formulations thereof, and especially its beneficial effects for gut.

### BACKGROUND OF THE INVENTION

Optimal digestive health is important to vitality and wellbeing throughout the entire life, and has got impact on the overall body's health, whereas gastrointestinal discomfort deteriorate quality of life.

Symptoms of gastrointestinal (GI) discomfort are often bloating, changes in intestinal mobility, transition time and pain in the GI tract, which may influence well-being. Gastrointestinal discomfort can be caused by distress, food composition as well as microbial distribution or disturbances within the intestine, which all can develop gastrointestinal disorders, including dyspepsia or irritations like irritable bowel syndrome (IBS) or inflammatory bowel disease (IBD).

About 20 percent of people, especially of the Western world, are affected with longer lasting gastrointestinal problems, like IBS, at least once in their life.

Many gastrointestinal disorders are caused by inflammatory processes and thus promoting digestive comfort can be achieved by anti-inflammatory nutritional or therapeutic maneuvers including down-regulation of gene expression involved in inflammation.

Standard therapeutic approaches include drugs like corticosteroids (cortisol, prednisone) and amino-salicylate derivatives, such as Sulphasalasine as well as immunomodulators, e.g. azathioprine, methotrexate, or natalizumab. These drugs exert anti-inflammatory effects, but have many severe side effects, which limit their use to an acute exacerbation. New drugs for inflammation reduction are of anti-oxidative character, like ethyl pyruvate, a synthetic alkyl ester of pyruvic ester.

Therefore, it is important to provide functional foods and dietary supplement ingredients with similar mode of action, but less adverse effects besides pharmaceutical drugs.

Alternative medicine and nutritional approaches include diets, probiotics (Escherichia coli Nissle 1917, Saccharomyces boulardii, bifidobacteria and lactic acid bacteria), prebiotics (barley, opuntia fibers, psyllium, oligofructose, inulin, modified starches, botanical or microbiotic derived gums), fish oils (omega-3 fatty acids) and herbal preparations (Frankincense (Boswellia serrata), Turmeric (Curcuma longa), Thyme spp., Chamomile, Cannabis), essential and aromatic oils, such as Peppermint, Chamomile, Clary sage, Fennel, Caraway, Thyme, and Lavender oil, and several nutritional supplements.

The focus of the present invention is to provide novel active ingredients for prevention and support of functional gastrointestinal discomfort and diseases, like inflammatory bowel illnesses and IBS applicable as functional foods, dietary supplement ingredients, and drugs.

### SUMMARY OF THE INVENTION

Surprisingly it was found that a composition comprising as active ingredient calcium pyruvates, and preferably calcium pyruvate monohydrate (CPM), or functionally active derivatives thereof has anti-inflammatory activity and IBS-relieving activity by inhibiting inflammation at molecular as well as at acting gene expression level.

### DETAILED DESCRIPTION OF THE INVENTION

Gastrointestinal disorders can be caused by inflammatory processes and amelioration can be achieved by anti-inflammatory nutrition or therapeutic drugs. Thus, therapeutic approaches include drugs like corticosteroids and amino-salicylate derivatives, as well as immunomodulators, which often provide anti-inflammatory effects as main benefit, but they may have many side effects, which limit their use. New drugs for inflammatory processes are in addition of anti-oxidative character, like ethyl pyruvate, a synthetic alkyl ester of pyruvic ester.

alpha-Keto carboxylic acids, including the pyruvic acid (2-Oxo-propanoic acid) and their derivatives, like esters and salts, called pyruvates are known for their anti-inflammatory efficacy (Fink M P (2008). Ethyl pyruvate. Current opinion in anaesthesiology 21(2):160-167).

On the other hand, pyruvic acid and pyruvates are reactive chemical compounds, which tend to dimerize or polymerize by aldolization. The common understanding is that the salt form with free pyruvate anion are less stable than ester forms of pyruvates. Apparently, pyruvate salts degrade faster than pyruvic acid esters, thus more recently pyruvate esters like ethyl pyruvate are strongly used in clinical research and salts are more and more neglected (Fink M P (2008). Ethyl pyruvate. Current opinion in anaesthesiology 21(2):160-167).

Additionally, simple pyruvate esters, like ethyl pyruvate, are not abandoned in nature or only in traces as fragrance component, thus they are of synthetic and non-physiological character, whereas pyruvic acid salts, like sodium and calcium pyruvates, are commonly found in living organisms, because pyruvates (in biochemistry synonym to pyruvic acid) are intermediates of sugar molecule metabolism and glycolysis.

Surprisingly, it was found with this invention that pyruvic acid salts of the alkaline and earth alkaline metal group (Ia and IIa), and especially CPM, in contradiction to prevailing scientific opinion is able to exert even better anti-inflammatory effects than pyruvic add esters and sufficient stability.

Anti-Inflammatory activity of pyruvate salts versus esters became obviously within a chemically induced colitis animal model. The experimental approach was to determine the effect of calcium pyruvate, especially as its monohydrate, and ethyl pyruvate at dosages of 20, 40 and 100 mg/kg body weight on the trinitrobenzene sulfonic acid (TNBS) model of colitis in rats, after a 7 days oral treatment, with main focus on colonic histology and inflammatory mediators. As key result was found that both pyruvates showed intestinal anti-inflammatory effects in TNBS-induced colitis. They were evident both histologically, with a recovery of the mucosal cyto-architecture and reduction of the neutrophil infiltration, and through the profile of inflammatory mediators (IL-1, IL-6, IL-17, IL-23, iNOS). However, calcium pyruvate appeared to be more effective than ethyl pyruvate and even better than the therapeutic drug Sulphasalazine (see examples 1b-1g). Remarkable in this context is the positive impact of calcium pyruvate on the mediators IL-17 and IL-23, which play a pivotal role in the etiology of inflammatory bowel processes (example 1e).

Basically, the animal studies were analogically performed as described in detail in the publication “Intestinal anti-inflammatory activity of the Serpylli herba extract in experimental models of rodent colitis” by Galvez J et al.; J Crohns Colitis. 2014:775-88.

Additionally, a relationship is nowadays seen between inflammatory bowel disease (IBD) and irritable bowel syndrome (IBS). Since most people with IBS appear physically healthy, a psychosomatic cause of IBS was rather considered in the past, but recently, “mini-inflammations” were discovered in the intestine of people with IBS, and related neuronal communication disorders in the nervous complex of the intestine were discussed as a cause. These inflammatory foci bring IBS in closer connection with IBD, which may also offer an anti-inflammatory treatment of irritable bowel similar to IBD (1. Bercik P, Verdu E F, Collins S M. Is irritable bowel syndrome a low-grade inflammatory bowel disease? Gastroenterol Clin North Am. (2005) Vol. 34, Issue: 2: 235-45//2. Mearin F, Perello A, Balboa A; Irritable bowel syndrome and inflammatory bowel disease: Is there a connection? Gastroenterologia y hepatologia (2009) Volume: 32, Issue: 5, Pages: 364-372//3. Quigley E M M, Shanahan F; Irritable Bowel Syndrome and Inflammatory Bowel Disease: Is There an Overlap? Practical Gastroenterology, November 2010, 31-37).

Thus, the effects of different doses of calcium pyruvate (CaPyr) or/and CPM and Gabapentin, a drug for IBS and as control herein became remarkable and very beneficial, as shown in an experimental model of IBS in rats induced by intracolonic administration of deoxycholic acid (DCA) on the abdominal withdrawal reflex to colorectal distension (CRD) and referred pain evaluated with Von Frey filaments as well as the gene expression of different markers evaluated in the colonic tissue by qPCR of IL-1β and COX-2.

Although the experimental findings of this invention show obviously positive effects of calcium pyruvate on inflammatory gastrointestinal processes, derivatives of calcium pyruvate, e.g. other hydrates (n=0-5×H₂O), especially its very stable and pure monohydrate, and reasonable combination and formulation ingredients, known from alternative medicine and nutritional approaches include diets, probiotics (Escherichia coli Nissle 1917, Saccharomyces boulardii, bifidobacteria and lactic acid bacteria), prebiotics (prebiotic oligosaccharides, including polydextrose, fructooligo-saccharides (FOS), xylooligosaccharides (XOS), galactooligosaccharides (GOS), and mannooligosaccharides (MOS), as well as lactulose and tagatose, barley fibers, psyllium, oligofructose, inulin, modified starches, botanical or microbiotic derived gums), fish oil (omega-3 fatty acids) and herbal preparations (frankincense (Boswellia serrata), Turmeric (Curcuma longa), Thyme spp., Chamomile, Cannabis), essential and aromatic oils, such as Peppermint, Chamomile, Clary sage, Fennel, Caraway, Thyme, and Lavender oil, and nutritional supplements do exert their merits or the formulations thereof are even synergistic in their mode of action.

Object of the present invention is to provide a novel functional ingredient that firstly is suitable for use in the entire range of inflammatory diseases, preferably with humans, and secondly has a sufficient stability and shelf-life. Local anti-inflammatory effects may be helpful along the entire digestive tract, as the case may be, in the treatment or prevention of inflammatory diseases. Gingivitis, parodontitis, pharyngitis, esophagitis and gastritis describe the inflammatory diseases of the upper part of the digestive tract. Lymphocytic colitis, ulcerative colitis, diverticulitis, duodenitis and Crohn's disease are, in addition to IBD and IBS, the best known representatives of inflammatory diseases of the intestinal region. Diseases of the intestinal region are the diseases belonging to the lower intestinal tract.

The present invention is not limited to human application only and shall be applicable to the group of mammalian animals, especially pets and livestock.

Surprisingly the agent calcium pyruvate (CaPyr), especially its monohydrate (CPM), was found having anti-oxidative, anti-inflammatory properties, which are based on molecular mechanisms including down-regulation of gene expression involved in inflammation, including IL-17, and IL-23. Additionally, its chemical stability is sufficient to exert the above efficacy.

The examples which follow further illustrate the invention, but should not be construed to limit the scope of the invention in any way.

The practice of the present invention will employ, unless otherwise indicated, conventional techniques of plant biology, chemistry, biochemistry, physiology and pharmacology which are within the skill of the art.

### EXAMPLES

### Example 1a

The TNBS model of rat colitis and effects of several doses of calcium pyruvate (CaPyr), ethyl pyruvate (EtPyr) and sulphasalzine (SAZ) on colonic macroscopic damage score, weight/length ratio, myeloperoxidase (MPO) activity and glutathione (GSH) content in TNBS experimental rat colitis was carried out and shown as follows. All these studies have been carried out in accordance with the ‘Guide for the Care and Use of Laboratory Animals’ as promulgated by the National Institute of Health.

Female Wistar rats (180-200 g) were randomly assigned to nine groups (n=10). Three groups received treatment with CaPyr (20, 40 and 100 mg/kg); other three received treatment with ethyl pyruvate (20, 40 and 100 mg/kg); and the remaining was treated with sulphasalazine (30 mg/kg). All compounds were dissolved in 1 ml of carboxymethylcellulose (0.2%) in water solution, and administered daily by oral gavage. An untreated TNBS control group and a non-colitic group were included for reference, which received the vehicle used to administer the test compounds. Colonic inflammation was induced in control and treated groups as generally described (see Galvez et al. 2014). Briefly, rats were fasted overnight, anesthetized with halothane and given TNBS dissolved in 0.25 ml of 50% ethanol (v/v) by means of a Teflon cannula inserted 8 cm through the anus. During and after TNBS administration, the rats were kept in a head-down position until they recovered from anaesthesia, and then returned to their cages. Rats from the non-colitic group were administered intracolonically 0.25 ml of phosphate buffered saline instead of TNBS. The treatments were given from the day of the colitis induction until the sacrifice of the rats with an overdose of halothane, seven days later. Animal body weights, occurrence of diarrhoea, and water and food intake were recorded daily throughout all the experiments. Once the animals were sacrificed, the colon was removed aseptically and placed on an ice-cold plate, longitudinally opened and cleaned from their luminal contents with cold saline. Afterwards, it was weighed and its length measured under a constant load (2 g). Each colon was scored for macroscopically visible damage on a 0-10 scale by two observers unaware of the experiment, according to the criteria described before (see Galvez et al. 2014). Colon samples (0.5 cm²) containing all the layers were taken from a region of the inflamed colon corresponding to the adjacent segment to the gross macroscopic damage and were fixed in 4% buffered formaldehyde for the histological studies. Equivalent colonic segments were also obtained from the non-colitic group. The colon was subsequently minced, aliquoted and kept frozen at −80° C. until biochemical determinations and RNA extraction was performed.

Histological Studies

Cross-sections were selected and embedded in paraffin. Full-thickness sections of 5 μm were obtained at different levels and stained with haematoxylin and eosin. The histological damage was evaluated by a pathologist observer, who was blinded to the experimental groups, according to the criteria previously described by Galvez.

Biochemical Determinations in Colonic Tissue

Myeloperoxidase (MPO) activity was measured according to the technique described previously (Krawiszet al., 1984); the results were expressed as MPO units per gram of wet tissue; one unit of MPO activity was defined as that degrading 1 μmol hydrogen peroxide/min at 25° C. Total glutathione (GSH) content was quantified with the recycling assay described by Anderson (1985), and the results were expressed as nmol/g wet tissue. In order to evaluate tissue IL-1β levels, the colonic samples were homogenized in phosphate buffered saline supplemented with 0.1% sodium dodecyl sulfate (SDS), 0.1% sodium deoxycholate, 1% Triton X-100 and protease and phosphatase inhibitors (aprotinin, leupeptin and phenylmethylsulfonyl fluoride). The cytokine was quantified by enzyme-linked immunoabsorbent assay (R&D Systems Inc., Minneapolis, Minn., USA) and the results were expressed as pg/g wet tissue.

Analysis of gene expression in colonic samples by RT-qPCR Total RNA from colonic samples was isolated using Trizol® (Thermo Fisher Scientific Inc., Waltham, Mass. USA) following the manufacturer's protocol. All RNA samples were quantified with the Thermo Scientific NanoDrop™ 2000 Spectrophotometer (Thermo Fisher Scientific Inc., Waltham, Mass. USA) and 2 pg of RNA were reverse transcribed using oligo(dT) primers (Promega, Southampton, UK).

Real time quantitative PCR amplification and detection was performed on optical-grade 48 well plates in an Eco™ Real-Time PCR System (Illumina, CA, USA) with 20 ng of cDNA, the KAPA SYBR® FAST qPCR Master Mix (Illumina, CA, USA) and specific primers at their annealing temperature (Ta). To normalize mRNA expression, the expression of the housekeeping gene, glyceraldehyde-3-phosphate dehydrogenase (GAPDH) was measured. The mRNA relative quantitation was calculated using the ΔΔCt method.

Statistics

All results are expressed as the mean±SEM. Differences between means were tested for statistical significance using a one-way analysis of variance (ANOVA) and post hoc least significance tests. Differences between proportions were analyzed with the chi-squared test. All results are expressed as the mean±SEM. Differences between means were tested for statistical significance using a one-way analysis of variance (ANOVA) with Tukey post-hoc test. Nonparametric data (macroscopic and microscopic scores) were analyzed by the Kruskal-Wallis test. All statistical analyses were carried out with the GraphPad Prism version 5.0 (GraphPad Software Inc., La Jolla, Calif., USA), with statistical significance set at P<0.05.

### Example 1b

Effects of several doses of calcium pyruvate (CaPyr), ethyl pyruvate (EtP)yr and sulphasalzine (SAZ) on colonic macroscopic damage score, weight/length ratio, myeloperoxidase (MPO) activity and glutathione (GSH) content in TNBS experimental rat colitis.

TABLE 1b

| | Damage | | | |
|---|---|---|---|---|
| Group | Score | Weight/length | MPO | GSH |
| (n = 10) | (0-10) | (mg/cm) | (mU/g tissue) | (nmol/g tissue) |
| Non-colitic | 0 | 80.3 ± 4.0 | 2.0 ± 0.3 | 1488 ± 141 |
| TNBS control | 7.9 ± 0.2 | 201.8 ± 15.2 | 83.8 ± 7.7 | 403 ± 58 |
| CaPyr (20 mg/kg) | 7.1 ± 0.4 | 186.0 ± 22.8 | 48.2 ± 7.2* | 389 ± 55 |
| CaPyr (40 mg/kg) | 5.7 ± 0.4* | 184.6 ± 25.1 | 48.8 ± 8.2* | 675 ± 67* |
| CaPyr (100 mg/kg) | 6.6 ± 0.6* | 198.9 ± 15.0 | 57.7 ± 11.5* | 768 ± 100* |
| EtPyr (20 mg/kg) | 6.6 ± 0.6* | 185.4 ± 31.1 | 58.1 ± 11.9 | 560 ± 74 |
| EtPyr (40 mg/kg) | 6.1 ± 0.4* | 172.0 ± 15.8 | 61.5 ± 9.8 | 715 ± 88* |
| EtPyr (100 mg/kg) | 6.0 ± 0.5* | 186.4 ± 13.2 | 54.7 ± 10.8* | 870 ± 124* |
| SAZ (30 mg/kg) | 6.7 ± 0.4* | 180.4 ± 16.9 | 82.8 ± 8.2 | 900 ± 118* |

Data are expressed as mean ± SEM.
*P < 0.05 vs. TNBS control group. All colitic groups statistically differ from non-colitic group (P < 0.05).

### Example 1c

Effects of several doses of calcium pyruvate (CaPyr), ethyl pyruvate (EtPyr) and sulphasalzine (SAZ) on the microscopic score according to Arribas et al. in TNBS experimental rat colitis.

TABLE 1c

| Group | Microscopic Damage Score |
|---|---|
| (n = 10) | (0-59) |
| Non-colitic | 0 |
| TNBS control | 35 ± 3 |
| CaPyr (20 mg/kg) | 27 ± 2 |
| CaPyr (40 mg/kg) | 18 ± 5* |
| CaPyr (100 mg/kg) | 13 ± 2* |
| EtPyr (20 mg/kg) | 25 ± 3 |
| EtPyr (40 mg/kg) | 17 ± 3* |
| EtPyr (100 mg/kg) | 23 ± 3* |
| SAZ (30 mg/kg) | 30 ± 5 |

Data are expressed as means ± SEM.
*P < 0.05 vs. TNBS control group.

### Example 1d

Effects of different doses of calcium pyruvate (CaPyr), ethyl pyruvate (EtPyr) and sulphasalazine (SAZ) in TNBS rat colitis on colonic IL-1β measured by ELISA and expressed as ng/g of tissue, and the mRNA expression of IL-1β, IL-6 and IL-12, quantified by real-time PCR and shown as fold increases.

TABLE 1d

| Group | IL-1β | IL-1β | IL-6 | IL-12 |
|---|---|---|---|---|
| (n = 10) | (ng/g tissue) | (fold increase) | (fold increase) | (fold increase) |
| Non-colitic | 5.4 ± 0.6 | 1.3 ± 0.03 | 1.01 ± 0.1 | 1.02 ± 0.06 |
| TNBS control | 1140.0 ± 90.1 | 9.00 ± 1.20 | 5.81 ± 0.25 | 2.34 ± 0.42 |
| CaPyr (20 mg/kg) | 1095.0 ± 165.0 | 3.90 ± 0.61* | 5.41 ± 1.01 | 1.09 ± 0.13 |
| CaPyr (40 mg/kg) | 765.0 ± 60.0* | 2.40 ± 0.61* | 3.20 ± 1.20* | 0.66 ± 0.18* |
| CaPyr (100 mg/kg) | 810.1 ± 120.0* | 4.80 ± 0.90* | 4.61 ± 1.01 | 1.62 ± 0.30 |
| EtPyr (20 mg/kg) | 975.0 ± 90.1 | 6.90 ± 1.50 | 5.60 ± 1.41 | 1.09 ± 0.13 |
| EtPyr (40 mg/kg) | 1140.0 ± 150.1 | 7.20 ± 1.20 | 5.60 ± 1.20 | 1.81 ± 0.48 |
| EtPyr (100 mg/kg) | 975.0 ± 211.7 | 10.21 ± 1.50 | 6.00 ± 1.01 | 1.62 ± 0.37 |
| SAZ (30 mg/kg) | 840.0 ± 120.0* | 9.70 ± 0.90 | 4.21 ± 0.80* | 1.59 ± 0.24 |

Data are expressed as means ± SEM (n = 10);
*P < 0.05 vs. TNBS control group.

### Example 1e

Effects of different doses of calcium pyruvate (CaPyr), ethyl pyruvate (EtPyr) and sulphasalazine (SAZ) in TNBS rat colitis on mRNA expression of IL-17, IL-23 and iNOS, quantified by real-time PCR and shown as fold increases.

TABLE 1e

| Group | IL-17 | IL-23 | iNOS |
|---|---|---|---|
| (n = 10) | (fold increase) | (fold increase) | (fold increase) |
| Non-colitic | 1.02 ± 0.03 | 0.99 ± 0.06 | 1.22 ± 0.06 |
| TNBS control | 2.16 ± 0.30 | 2.37 ± 0.27 | 19.82 ± 2.14 |
| CaPyr (20 mg/kg) | 0.84 ± 0.09* | 1.47 ± 0.24* | 10.37 ± 1.83* |
| CaPyr (40 mg/kg) | 0.63 ± 0.27* | 1.02 ± 0.21* | 8.85 ± 3.36* |
| CaPyr (100 mg/kg) | 1.83 ± 0.48 | 1.98 ± 0.30 | 9.15 ± 1.53* |
| EtPyr (20 mg/kg) | 1.17 ± 0.27* | 1.68 ± 0.39 | 16.17 ± 2.75 |
| EtPyr (40 mg/kg) | 1.74 ± 0.33 | 1.56 ± 0.27* | 12.20 ± 1.53* |
| EtPyr (100 mg/kg) | 2.34 ± 0.30 | 1.80 ± 0.21 | 22.88 ± 2.14 |
| SAZ (30 mg/kg) | 2.22 ± 0.54 | 1.26 ± 0.18* | 16.47 ± 2.75 |

Data are expressed as means ± SEM (n = 10);
*P < 0.05 vs. TNBS control group.

### Example 1f

Effects of different doses of calcium pyruvate (CaPyr), ethyl pyruvate (EtPyr) and sulphasalazine (SAZ) in TNBS rat colitis on mRNA expression of CINC-1, MCP-1 and ICAM-1, quantified by real-time PCR and shown as fold increases.

TABLE 1f

| Group | CINC-1 | MCP-1 | ICAM-1 |
|---|---|---|---|
| (n = 10) | (fold increase) | (fold increase) | (fold increase) |
| Non-colitic | 1.06 ± 0.04 | 0.98 ± 0.04 | 1.07 ± 0.01 |
| TNBS control | 5.46 ± 0.49 | 3.20 ± 0.21 | 4.79 ± 0.44 |
| CaPyr (20 mg/kg) | 3.59 ± 0.49* | 3.03 ± 0.49 | 1.64 ± 0.25* |
| CaPyr (40 mg/kg) | 2.93 ± 0.57* | 1.80 ± 0.37* | 0.95 ± 0.19* |
| CaPyr (100 mg/kg) | 3.50 ± 0.90* | 2.13 ± 0.41* | 2.77 ± 0.44* |
| EtPyr (20 mg/kg) | 4.40 ± 0.65 | 1.23 ± 0.37* | 1.39 ± 0.19* |
| EtPyr (40 mg/kg) | 4.40 ± 0.57 | 1.85 ± 0.29* | 2.27 ± 0.38* |
| EtPyr (100 mg/kg) | 4.40 ± 0.90 | 2.21 ± 0.29* | 2.39 ± 0.50* |
| SAZ (30 mg/kg) | 4.89 ± 0.82 | 2.62 ± 0.41* | 2.90 ± 0.88* |

Data are expressed as means ± SEM (n = 10);
*P < 0.05 vs. TNBS control group.

### Example 1g

Effects of different doses of calcium pyruvate (CaPyr), ethyl pyruvate (EtPyr) and sulphasalazine (SAZ) in TNBS rat colitis on mRNA expression of MUC-2 and TFF-3 quantified by real-time PCR and shown as fold increases.

TABLE 1g

| Group | MUC-2 | TFF-3 |
|---|---|---|
| (n = 10) | (fold increase) | (fold increase) |
| Non-colitic | 1.02 ± 0.08 | 0.99 ± 0.05 |
| TNBS control | 0.26 ± 0.02 | 0.36 ± 0.05 |
| CaPyr (20 mg/kg) | 0.50 ± 0.14* | 0.84 ± 0.23* |
| CaPyr (40 mg/kg) | 0.53 ± 0.16* | 0.67 ± 0.17* |
| CaPyr (100 mg/kg) | 0.56 ± 0.11* | 0.99 ± 0.19* |
| EtPyr (20 mg/kg) | 0.39 ± 0.09 | 0.59 ± 0.16 |
| EtPyr (40 mg/kg) | 0.70 ± 0.08* | 1.01 ± 0.05* |
| EtPyr (100 mg/kg) | 0.50 ± 0.11* | 0.96 ± 0.11* |
| SAZ (30 mg/kg) | 0.53 ± 0.14* | 0.91 ± 0.12* |

Data are expressed as means ± SEM (n = 10);
*P < 0.05 vs. TNBS control group.

### Example 2

Effects of Calcium Pyruvate Monohydrate (CPM) in an experimental model of irritable bowel syndrome (IBS) in rats induced by intracolonic administration of deoxycholic acid (DCA) was carried out and shown as follows. All these studies have been carried out in accordance with the ‘Guide for the Care and Use of Laboratory Animals’ as promulgated by the National Institute of Health.

Male Sprague Dawley rats (240-320 g) were administered DCA once daily on 3 consecutive days, and then divided into the different experimental groups (n=8): three received orally and daily CPM (40 and 100 mg/kg) or Gabapentin (70 mg/kg); a non IBS and an untreated control IBS group were also included. One and two weeks after, abdominal withdrawal reflex to colorectal distension (CRD) was semi quantitatively scored. Also the referred pain was evaluated with Von Frey filaments. After two weeks of treatment, all rats were sacrificed and the expression of different markers IL-1β and COX-2 evaluated in the colonic tissue by qPCR.

TABLE 2

| Group | CRD | AUC | qPCR IL-1β | qPCR COX-2 |
|---|---|---|---|---|
| (n = 8) | (units) | (arbitrary units) | (fold increase) | (fold increase) |
| Non-IBS | 0.14 ± 0.0^{a} | 54.55 ± 19.93^{a} | 1.15 ± 0.23^{a} | 1.02 ± 0.15^{a} |
| IBS control | 2.21 ± 2.5^{b} | 188.83 ± 27.30^{b} | 19.78 ± 3.45^{b} | 5.00 ± 1.16^{b} |
| CPM (40 mg/kg) | 1.30 ± 0.14^{c} | 104.65 ± 20.48^{a} | 7.82 ± 2.07^{c} | 2.47 ± 0.58^{c} |
| CPM (100 mg/kg) | 0.70 ± 0.14^{a,c} | 63.70 ± 22.75^{a} | 5.52 ± 1.15^{c} | 1.60 ± 0.36^{c} |
| Gabapentin (30 mg/kg) | 0.25 ± 0.1^{a} | 27.30 ± 6.83^{a} | 8.28 ± 1.38^{c} | 1.74 ± 0.36^{c} |

Data are expressed as means ± SEM (n = 8);
*P < 0.05 vs. control or other groups (with different letters).

One and two weeks of treatment resulted in reduced CRD scores values for the treated groups compared with the IBS control. Also, the referred pain was attenuated in those groups treated with CPM and gabapentin. The IBS process was associated with altered expression of the different markers studied and the treatments were associated with a restoration in the expression of IL-1β and COX-2.

In conclusion, Calcium pyruvate monohydrate (CPM) is able to exert beneficial effects in the experimental model of IBS, with a similar efficacy to that showed by the standard therapy Gabapentin and this effect was associated with an improvement of the altered immune response clearly involved in IBS.

### Example 3

### Formulations

### Example 3a

### Manufacture of a Water-Soluble Formula for Use in Drinks

Liquid formulations are delivery forms for application of 20 mg to 5,000 mg per serving of pyruvic acid salts, especially calcium pyruvates. The calcium pyruvates' preparation is fully water-soluble and can be added to drink concentrates, dietetic foods and dairy products in concentrations ranging from 1 to 10%. Thus, the pyruvate salts are blended with prebiotic or non-digestible carbohydrates, and fillers like maltodextrin, fructose or other, ideally sugar-free, completely water-soluble, with low glycemic indices, such as hydrolysed proteins (Gelitasol etc.). So the mentioned substances are used as adjuvants, and these preparations can also be used in food products, dietary foods for special medical purposes and drinks for inflammatory intestinal diseases or IBS. One to five, more specifically two to three servings a day (20 mg to 25 grams/day), depending on the mammalian species, kind of disease and their severity are recommended.

### Example 3b

### Pectin Lozenges

40 grams of standard commercial citrus pectin and 2 grams of trisodium citrate were mixed with 100 grams of Isomalt sugar substitute. This mixture was stirred into 200 ml water and, stirring continuously, heated until all the pectin had dissolved. A further 475 g Isomalt and 260 g fructose syrup were added and the mixture boiled until the dry matter content reached approx. 80%. 5-50 g of an alkaline or earth alkaline pyruvate and colours and flavourings were now added. Approx. 17 ml of a 50% citric acid solution was added to achieve a pH of 3.4-3.5. The mixture was poured into moulds at a temperature of approx. 95° C. to obtain 2 g lozenges. About 500 lozenges weighing 2 grams each can be manufactured in this way. One pectin lozenge contains approx. 10-100 mg of the pyruvate salts.

### Example 3c

### Gum Arabic-Based Lozenges

15 kg of a 33% gum arabic, 5% sorbitol, 20% water and 42% maltitol solution was produced in a stirrer at 65° C. 50-450 grams of an alkaline or earth alkaline pyruvate and 50 grams of ethyl pyruvate, plus flavourings and (if required) sweeteners are stirred into this solution. After complete homogenisation, the mixture is poured into starch moulds. After drying at 50° C., the final weight of the lozenges is set to 2.0 g, each containing approx. 10-100 mg of pyruvate. The lozenges are separated from the starch, treated with a releasing agent and packaged. Approx. 5000 lozenges are obtained.

### Example 3d

### Chewing Gums

100 g chicle is powdered, mixed with 300 g Isomalt sugar substitute and heated in an evaporating dish until it softens. 5 g of a mixture of calcium pyruvate monohydrate and alkaline or earth alkaline pyruvate or ethyl pyruvate is added and the mixture is mixed thoroughly, placed on a starched surface and kneaded to homogeneity. Flavourings may also be added during the previous step. The mixture is then rolled out into thin sheets and cut into flat sticks whilst still warm. The mixture is prevented from sticking to the surface by using a little starch powder. The chewing gum sticks should be 2 grams in weight and contain approx. 25 mg of the pyruvates.

### Example 3e

### Manufacture of a Pharmaceutical Tablet Composition

A defined amount of alkaline or earth alkaline pyruvate is directly pressed into tablets using the following recipe for a single tablet.
300 mg calcium pyruvate monohydrate
160 mg microcrystalline cellulose
25 mg sodium carboxymethylcellulose
10 mg highly dispersed silicon dioxide
5 mg magnesium stearate

### Example 3f

### Manufacture of a Pharmaceutical Hard Gelatine Capsule Composition

A defined amount of alkaline or earth alkaline pyruvate is directly filled into capsules using the following recipe per single capsule.
100 mg calcium pyruvate monohydrate
100 mg sodium pyruvate
50 mg microcrystalline cellulose
2 mg highly dispersed silicon dioxide

1-10. (canceled)

11. A method for treating inflammatory diseases of the intestinal tract of mammalians by using at least one pyruvic acid salt whereas the water content of the salt is 0 to 5 molecules.

12. The method according to claim 11, whereas the water content of the salt is 0 to 2.5 molecules.

13. The method according to one of the preceding claims, whereas the at least one pyruvic acid salt is preferably is calcium pyruvate monohydrate.

14. The method according to one of the preceding claims, whereas the inflammatory diseases of the intestinal tract is selected is irritable bowel syndrome (IBS).

15. A composition for use in a method for treating inflammatory diseases of the intestinal tract of mammalians by using calcium pyruvate monohydrate,

16. The composition according to claim 15, whereas the inflammatory diseases of the intestinal tract is irritable bowel syndrome (IBS).

17. The composition according to one of the preceding claims 15 and 16 comprising a prebiotic selected from the group of water-soluble carbohydrates.

18. The composition according to claim 17 comprising a prebiotic selected from the group of prebiotic oligosaccharides.

19. The composition according to claim 18 comprising a prebiotic selected from the group of polydextrose, fructooligo-saccharides (FOS), xylooligosaccharides (XOS), galactooligosaccharides (GOS), and mannooligosaccharides (MOS).

20. The composition according to one of the preceding claims 15 to 19 comprising calcium pyruvate monohydrate with a water content of 0-2.5 molecules H₂O and inulin-type prebiotic, a fructooligo-saccharides (FOS).

14. The method according to one of the preceding claims, whereas the inflammatory diseases of the intestinal tract is irritable bowel syndrome (IBS).

15. A composition for use in a method for treating inflammatory diseases of the intestinal tract of mammalians by using calcium pyruvate monohydrate.

16 The composition according to claim 15, whereas the inflammatory disease of the intestinal tract is irritable bowel syndrome (IBS).

17. The composition according to one of the preceding claims 15 and 16 comprising a prebiotic selected from the group of water-soluble carbohydrates.

18. The composition according to claim 17 comprising a prebiotic selected from the group of prebiotic oligosaccharides.

19. The composition according to claim 18 comprising a prebiotic selected from the group of polydextrose, fructooligo-saccharides (FOS), xylooligosaccharides (XOS), galactooligosaccharides (GOS), and mannooligosaccharides (MOS).

20. The composition according to one of the preceding claims 15 to 19 comprising calcium pyruvate monohydrate with a water content of 0-2.5 molecules H₂O and inulin-type prebiotic, a fructooligo-saccharides (FOS).
